(19) United States
(12) Patent Application Publication
(10) Pub. No.: US 2013/0025598 A1
(43) Pub. Date: Jan. 31, 2013

(54) OXYGEN DELIVERY SYSTEM

(71) Applicant: Denise Eiseman, Littleton, CO (US)

(72) Inventor: Denise Eiseman, Littleton, CO (US)

(21) Appl. No.: 13/557,979

(22) Filed: Jul. 25, 2012

Related U.S. Application Data
(60) Provisional application No. 61/511,423, filed on Jul. 25, 2011.

(51) Int. Cl. A61M 16/12; A61M 16/00; A61M 16/20

(52) U.S. Cl. 128/204.23; 128/204.18; 128/205.24

(57) ABSTRACT

The present disclosure is directed to an oxygen delivery system that distributes oxygen-enriched air around a patient's head and face without requiring a physical connection to the patient. The oxygen is distributed by the system through one or more fluid outputs, which are operatively connected to an oxygen supply line either directly or indirectly through at least one fluid transmission line, manifold or plenum. This directed oxygen mixes with the air surrounding the patient to produce an oxygen-enriched environment at the exit side of the fluid outputs.

### CROSS REFERENCE TO RELATED APPLICATION

The present application claims the benefit of and priority, under 35 U.S.C. §119(e), to U.S. Provisional Application Ser. No. 61/511,423, filed Jul. 25, 2011, entitled “OXYGEN DELIVERY SYSTEM,” which is hereby incorporated herein by reference in its entirety for all that it teaches and for all purposes.

### FIELD OF THE DISCLOSURE

The present disclosure relates to a fluid delivery apparatus, and more particularly to an oxygen delivery system.

### BACKGROUND

Human beings and animals in general require oxygen to survive. Although air is comprised of approximately 78% nitrogen, 21% oxygen, and 1% other gasses, it is the oxygen in air that sustains life. When oxygen is breathed into the lungs it is then distributed throughout the body via red blood cells and the circulatory system. Oxygen is then used by the body's cells, tissues, and organs to convert food into energy and heat. This energy is critical to life, and without oxygen, the body could not create the required heat or energy necessary to survive.

Oxygen is particularly critical for those who suffer lung, health, or heart problems. For example, patients who suffer from heart or lung problems cannot adequately pump enough oxygen-rich blood throughout their circulatory system to sustain a high quality of life. In these cases, supplemental oxygen is required to enjoy a higher quality of life, and in some instances survive.

Supplemental oxygen is an increased level of oxygen (above the normal levels of oxygen found naturally in the environment) that a body with health problems may require to operate as would a healthy body. By breathing supplemental oxygen, a patient will increase the amount of oxygen content that passes throughout their body and as a result see an increase in body function, rehabilitation, and rejuvenation.

Currently, supplemental oxygen delivery systems require the use of invasive or uncomfortable apparatuses to administer oxygen to a patient. Specifically, low-flow oxygen is delivered to a patient by two primary (but different) apparatuses, namely, the Nasal Cannula and the Face Mask.

The Nasal Cannula attaches to a patient's face and head via a hollow flexible supply tube that runs from an air source to a manifold which rests under the patient's nose. Two separate hollow tube extensions, or cannula, enter the nasal cavity (usually perpendicular to the supply tube air flow), one tube per cavity, from the tube manifold and direct oxygen flow into the nose.

As a result, nasal cannula are uncomfortable to wear and are especially disruptive during sleep. Due to the sensitive nature of the nasal cavity, some wearers of nasal cannula experience nose bleeds and irritation. Moreover, the nasal cannula limits a patient's range of motion, and can even cause minor air constriction or strangulation during a sleep state or irregular movement. In addition, the nasal cannula cannot provide oxygen to a patient whose nasal cavity is blocked or one who tends to breathe through his or her mouth.

Face masks are usually attached to a patient by use of an elastically adjustable strap that wraps around the head of the patient wearing the device. Typically, the face mask is a tent-like structure that provides an air chamber around the mouth and nasal area with a hollow supply tube extending to the air source. In this approach, the face mask's structure acts like a manifold that essentially provides a continuous flow of air around both the mouth and nose of the patient wearing the device. The face mask may cover the entire face, the mouth and nasal area, or only the mouth or nasal area of a patient.

In addition to the discomfort, fitting issues, and limited range of motion commonly associated with the face mask, other major problems exist with this particular device. For example, a face mask can cause skin irritation due to allergic reaction or prolonged use. Furthermore, a face mask cannot be worn by those who suffer from burns or other facial injuries. In other examples, face masks must be removed to allow a patient the ability to talk or eat. As can be appreciated, the face mask can induce a state of claustrophobia in some individuals. Moreover the face mask may cause nausea due to the smell of the plastic. Another problem with face masks is they cannot be easily adapted to treat non-human animals.

### SUMMARY

It is a long felt need in the field of oxygen delivery systems to provide a system capable of delivering supplemental oxygen to a patient without the associated discomfort of contact- based invasive nasal cannula and/or face masks and other attached devices. The following disclosure describes a device that can deliver adequate quantities of supplemental oxygen to a patient while avoiding all of the problems associated with the prior art. Throughout this disclosure, use of the word patient refers to any animal, human or non-human, in any condition of health and development.

The present disclosure is directed to an oxygen delivery device that distributes oxygen-enriched air around a patient's head and face without requiring a physical connection to the patient. In some embodiments, the device receives an ample supply of oxygen from a standard oxygen supply source, or other oxygen supply means, via one or more fluid supply lines. This fluid supply line can be flexible or otherwise operatively connected to an oxygen distribution area located adjacent to, on, or inside the device. The oxygen distribution area may be configured as a manifold or plenum. Additionally or alternatively, the oxygen distribution area may be configured as one or more sections of tubing. Also attached to the oxygen distribution area are one or more nozzles that direct oxygen from the oxygen distribution area toward a patient who is situated on the exit side of the nozzle flow. As the oxygen exits the nozzles, it mixes with the air surrounding the patient to create an oxygen-enriched environment.

The device can operate as a simple system requiring little to no adjustment, or in an alternative embodiment, one that allows for the full adjustment of the flow and/or direction of the oxygen distributed by the system either manually, automatically, or both. This control can be achieved through mechanical, analog, or digital mechanisms.

It is one aspect of the present disclosure to provide an oxygen distribution system that is easily useable, especially in a non-hospital or healthcare environment. Accordingly, an oxygen distribution system is described which employs an oxygen delivery device that can be attached to a wall, bed, table, ceiling, or any other object in proximity to a patient. In some embodiments, the oxygen delivery device is connected to an object via an articulating arm and clamp system. Other mechanical connecting members may, however, be used to connect the oxygen delivery device to at least one object proximate to the patient.

It is another aspect of the present disclosure to provide either manual or automated control mechanisms over the way in which oxygen is delivered by the oxygen delivery system. For instance, one or more oxygen, pulse, or similar sensors may be placed in proximity to or within the oxygen-enriched environment. As can be appreciated, a fluid flow path may be created between the oxygen supply source and the patient. In some embodiments, the one or more sensors may be placed in the fluid flow path between the oxygen supply source and the patient. Readings from these sensors can be provided to an automated control mechanism that controls the rate at which oxygen is dispensed from the oxygen delivery device, the direction in which oxygen is dispensed, or any other characteristic of the fluid delivery to help control characteristics of the oxygen-enriched environment.

In some embodiments, the sensors may comprise biometric sensors that are in communication with a patient. Among other things, these biometric sensors (e.g., oxygen saturation rate, pulse measuring, breathing monitors, and the like) may determine a pulse rate associated with a patient in the oxygen-enriched environment. The biometric data obtained by the biometric sensors may be used to control aspects of the oxygen delivery system, including but not limited to alarms, fluid flow characteristics, oxygen concentration levels, and the like. Accordingly, and in any or all of the disclosed embodiments, a control feedback loop may be created to facilitate better user control over the operation of the oxygen delivery system. In the control feedback loop, any type of known control electronics or components may be used without departing from the scope of the present disclosure.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

It shall be understood that the term “means” as used herein shall be given its broadest possible interpretation in accordance with 35 U.S.C., Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the invention, brief description of the drawings, detailed description, abstract, and claims themselves.

The term “attach” and variations thereof, as used herein, refers to any method, technique, or process to secure one thing to another. The attachment means may be removable, permanent, or semi-permanent. Typical attachments may include securing by adhesive, magnetic attraction, interference fit, fastener connections, tongue-in-groove, dovetail, press-fit, welding, ultrasonic welding, and the like. Accordingly, the terms “join,” “connect,” “adhere,” “fix,” “affix,” “append,” “glue,” “screw,” and “fasten” can be used interchangeably herein.

The preceding is a simplified summary of the disclosure to provide an understanding of some aspects of the disclosure. This summary is neither an extensive nor exhaustive overview of the disclosure and its various aspects, embodiments, and/or configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and/or configurations of the disclosure are possible utilizing, alone or in combination, one or more of the features set forth above or described in detail below.

### BRIEF DESCRIPTION OF THE DRAWINGS

The accompanying drawings, which are incorporated in and constitute a part of the specification, illustrate embodiments of the disclosure and together with the general description of the disclosure given above, and the description of the drawings given below, serve to explain the principals of this disclosure.

FIG. 1 is an oxygen delivery system in an operating environment in accordance with embodiments of the present disclosure;

FIG. 2 is a first perspective view of an oxygen delivery device in accordance with embodiments of the present disclosure;

FIG. 3 is a second perspective view of an oxygen delivery device in accordance with embodiments of the present disclosure;

FIG. 4 is a first exploded perspective view of an oxygen delivery device including fluid transmission lines in accordance with embodiments of the present disclosure; and

FIG. 5 is a second exploded perspective view of an oxygen delivery device including a plenum area in accordance with embodiments of the present disclosure.

It should be understood that the drawings are not necessarily to scale. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

The present disclosure is directed to an oxygen delivery device and system incorporating such a device. In some embodiments, the oxygen delivery device is used to distribute oxygen-enriched air around or about a patient's head and face without requiring a physical connection to the patient.

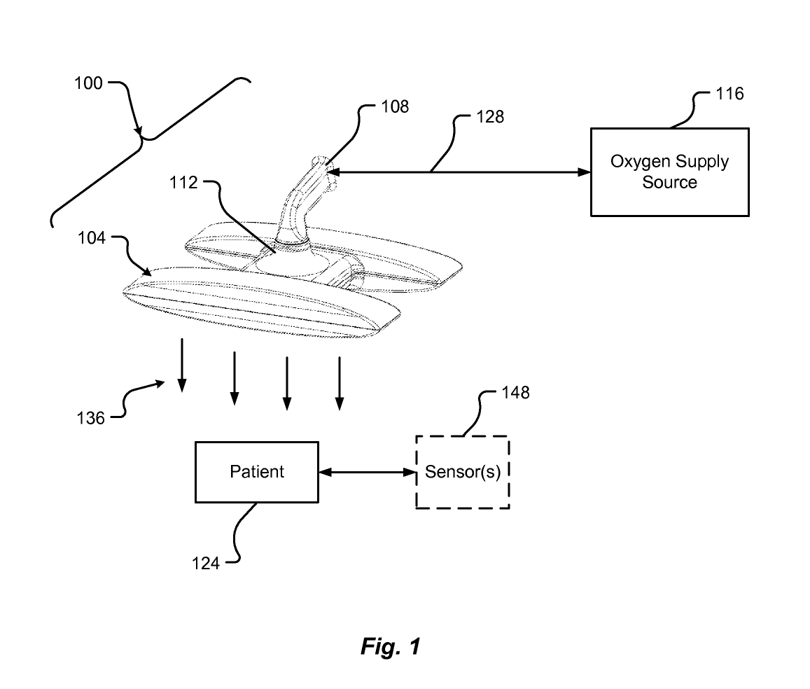

Referring to FIGS. 1-5, an oxygen delivery system is depicted in accordance with embodiments of the present disclosure. The oxygen delivery system may include one or more components that are used to deliver concentrated oxygen to a predetermined location, thereby creating an oxygen-enriched environment. The oxygen-enriched environment created by the oxygen delivery system can represent a location of increased oxygen saturation as compared to ambient air (e.g., the environment surrounding the oxygen-enriched environment). In some embodiments, a patient may position themselves within the oxygen-enriched environment such that the patient can experience the benefits of increased oxygen levels without requiring a facemask or nasal cannula.

Referring now to FIG. 1-3, additional details of embodiments of an oxygen delivery system will be described. In some embodiments, the oxygen delivery system includes an oxygen delivery device 100 that receives oxygen created by an oxygen supply source 116 via one or more fluid supply lines 128. The one or more fluid supply lines 128 may be oriented inside, adjacent to, and/or outside of the device 100. In some embodiments, the oxygen supply source 116 may comprise an oxygen concentrator. In some embodiments, the oxygen supply source 116 may be an oxygen supply connection, where oxygen is provided via some other means (e.g., a hospital oxygen connection). In any case, the oxygen supply source 116 may supply oxygen to the oxygen delivery device 100 via one or more fluid supply lines 128. Although described herein as ideally suited for providing an oxygen-enriched environment, it is anticipated that embodiments of the device 100 may receive some other gas, or combination of gasses, from one or more sources other than an oxygen condenser unit 116 as disclosed.

In some embodiments, the device 100 is mounted to a patient's bed via an arm 108 and clamp system. The arm 108 may be configured to at least partially contain the one or more fluid supply lines 128. In one embodiment, the arm 108 may include a receptacle to contain and/or conceal the one or more fluid supply lines 128. As can be appreciated, the arm 108 and clamp system may not be necessary if the device 100 is integrated into a support structure and the oxygen-enriched environment is to remain static. However, it may be desirable to have the ability to change or alter the position of the oxygen-enriched environment, in which case the use of the arm 108 attached to some articulating device may be beneficial. In one embodiment, the arm 108 may include features to allow the device 100 to be positioned in one or more locked positions. For example, the housing 104 of the device 100 may be configured to swivel, or rotate, about an axis created by the arm 108. Accordingly, the rotatable connection may incorporate the use of friction features and/or detents to allow movement into a number of positions. The device may be locked in a specific or general position with a detent system, clamp arrangement, and the like.

In some embodiments, the device 100 is connected to the arm 108 via a mount plate 112 on the housing 104 of the device 100. This mount plate 112 may utilize custom or standard bolt patterns (e.g., VESA, etc.) to interface with custom arms and supports or standard articulating or fixed arms that are available off-the-shelf. This standard mount or bolt pattern may be used at the other end of the arm 108 to connect to a wall, pedestal mount, articulating arm, static arm, ceiling, surface, and the like. In some embodiments, the mount plate 112 may be fixedly attached to the housing 104 and the arm 108 may be operatively connected to the fixedly attached mount plate via one or more locking features, plug and receptacle connection, welding, gluing, interference fits, and the like. By mounting the device 100 to an articulating arm 108, the entire unit 100 can be adjusted to different angles to achieve the optimal oxygen-enriched environment for the patient 124.

FIG. 1 shows the device 100 in an operating environment in accordance with embodiments of the present disclosure. In some embodiments, the device 100 may be positioned to direct oxygen 136 into the oxygen-enriched environment that is created adjacent to a patient 124. The oxygen-enriched environment may be created by expelling oxygen 136 in the general direction of the patient's 124 head and/or face. It is anticipated that the device 100 may be moved to a stowed position when not used by the patient 124. For example, the device 100 may be moved away from the patient's 124 head and/or face and stored in an area free from patient movement. In one embodiment, the device 100 may be stored above the patient 124. In another embodiment, the device 100 may be stowed against the surface of a wall or support. In yet another embodiment, the housing 104 of the device 100 may rest flush with or close to a wall of a room or other vertical surface thereby taking up less usable room space.

In accordance with embodiments of the present disclosure, the device 100 may be configured to receive oxygen via an oxygen supply source 116 and/or oxygen supply means. The oxygen may be transmitted from the oxygen supply source 116 to the device 100 via one or more fluid supply lines 128 connected therebetween. In one embodiment, the oxygen transmitted from the oxygen supply source 116 to the device 100 may create a fluid flow path within one or more fluid supply lines 128. The flow of oxygen within the one or more fluid supply lines 128 may be monitored and/or measured. For example, at least one of a flow and pressure sensor may be arranged in the fluid flow path to detect pressure and/or flow changes in the one or more fluid supply lines 128. Detection of a change in pressure and/or flow may be used to alert a patient 124 of changes to the oxygen output by the oxygen condenser unit 116 and/or the device 100.

In one embodiment, one or more biometric sensors 148 may be used to monitor biological input provided by at least one patient 124. For example, the one or more biometric sensors 148 may be configured to obtain a pulse rate and/or oxygen saturation levels (e.g., SpO2) from a patient 124. It is anticipated that this SpO2 information may be obtained by continuous and/or interval measurement samples. In the event that two or more patients 124 are positioned in the oxygen-enriched environment created by the device 100, individual biometric sensors may be used for each patient 124. Additionally or alternatively, the one or more sensors disclosed herein may be used to automatically control the oxygen output to one or more patients 124. Further still, the sensors 148 may communicate with a control mechanism and/or an alarming mechanism via wired and/or wireless communication protocols known or yet to be developed.

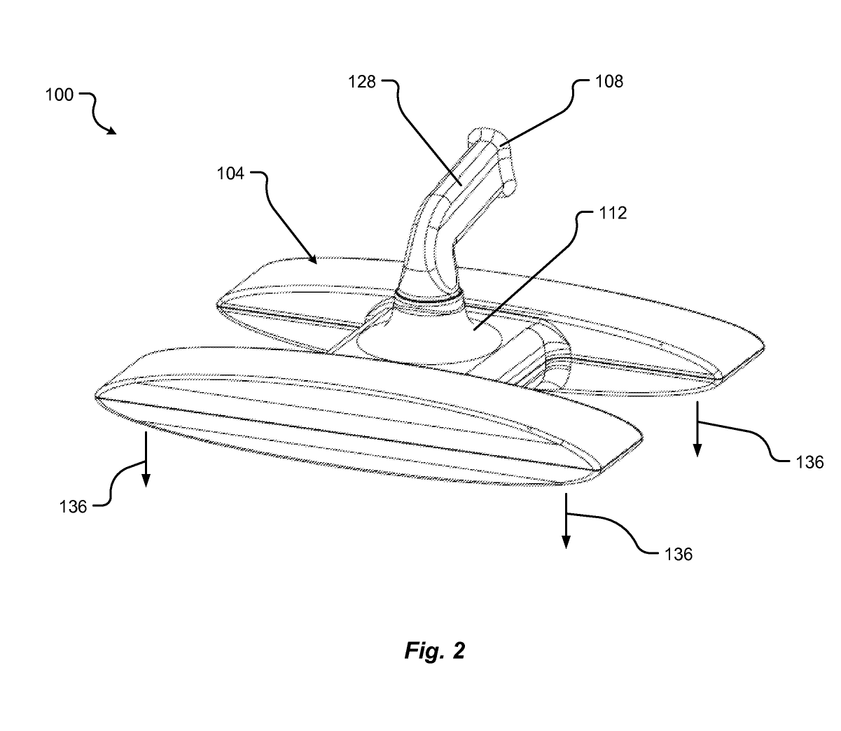

Referring now to FIG. 2, a first perspective view of the device 100 is shown, which may correspond to an operational position of the device 100. As can be seen in FIG. 2, the device 100 includes a housing 104, a fluid supply line 128, and a mounting plate 112. The device 100 is connected to an articulating arm 108 or other physical support which can be connected to a pedestal mount or other mechanical interface. It is anticipated that the device 100 directs oxygen 136 into an oxygen-enriched environment adjacent to the device 100.

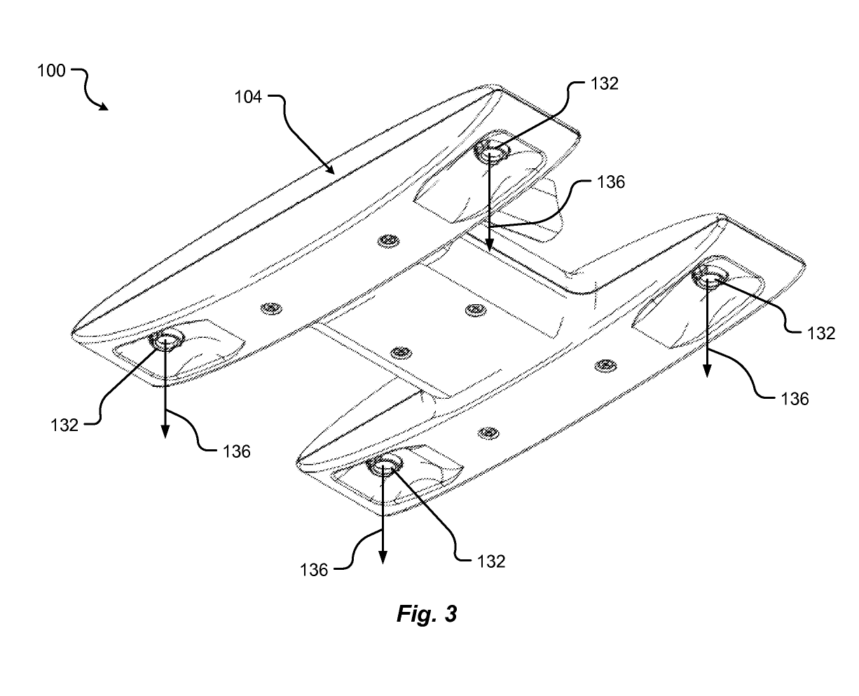

FIG. 3 shows a second perspective view of the device 100, including one or more oxygen outputs 132. The device 100 directs oxygen 136 into the oxygen-enriched environment by first receiving oxygen at the fluid supply line 128 and then distributing the received oxygen among a plurality of oxygen outputs 132. As will be discussed in further detail herein, the oxygen may be distributed among the plurality of oxygen outputs 132 via a plurality of fluid transmission lines or via a common manifold or plenum. In either configuration, the oxygen provided from the fluid supply line 128 to the oxygen outputs 132 can be expelled in the general direction of the patient's head and face 124 (see FIG. 1). Because the device 100 is not directly attached to the patient, the patient is free to move within the oxygen-enriched environment created by the device 100 without suffering the discomfort associated with wearing nasal cannula, face masks, or other attached equipment. In addition, because oxygen is generally heavier than air, the distributed oxygen 136 will naturally cascade from the oxygen outputs 132 over the patient 124 who lies beneath the device 100 creating this oxygen-enriched environment.

In some embodiments, the oxygen outputs 132 may comprise one or more paths in the housing 104, restrictions in the housing 104, manifolds, openings, and/or nozzles. The type of oxygen output 132 selected for use in the device 100 may vary depending upon the desired characteristics of the oxygen-enriched environment. In particular, the manner by which oxygen 136 is directed toward the patient 124 (see FIG. 1) can be accomplished via different embodiments of the device 100 and oxygen output 132 elements.

In some embodiments, the housing 104 shown could be increased in width to effectively extend over two or more patients providing a larger oxygen-enriched environment. Stated another way, the dimensions of the housing 104 and device 100 can be selected to accommodate a number of different environments, and all such modifications are considered to be within the scope of the present disclosure.

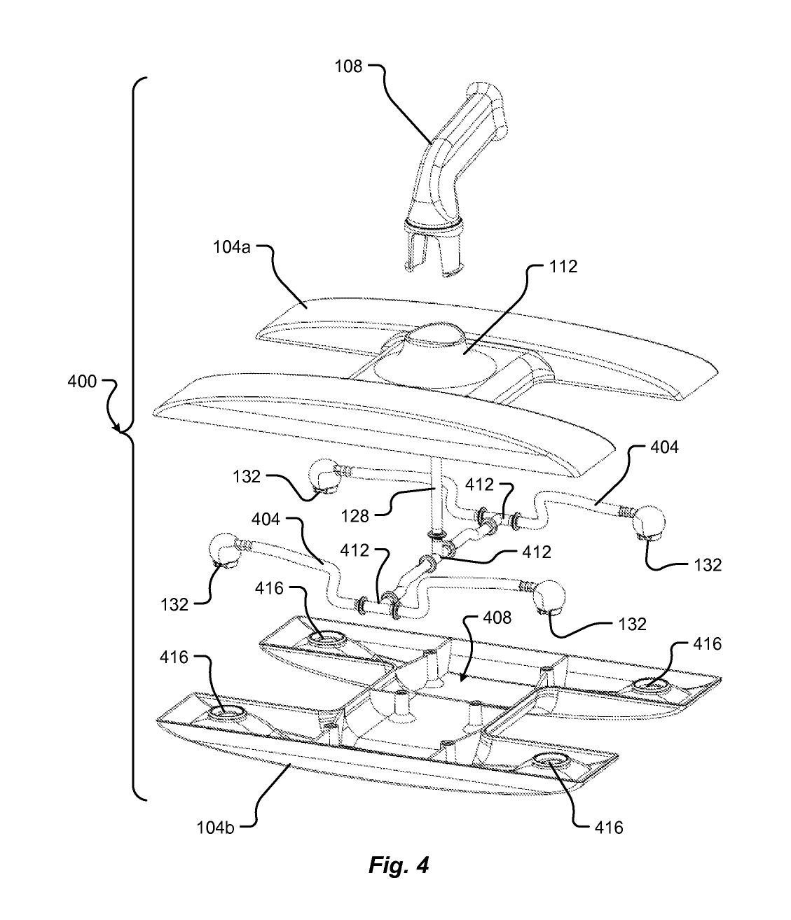

Referring now to FIG. 4, a first exploded perspective view of the oxygen delivery device 400 is shown in accordance with embodiments of the present disclosure. In particular, the device 400 depicted in FIG. 4 employs a plurality of fluid transmission lines 404 as a mechanism for transporting oxygen from the fluid supply line 128 to the oxygen outputs 132. In some embodiments, each of the fluid transmission lines 404 comprises a proximate and distal end. In this case, the proximate ends of the fluid transmission lines 404 are connected to the fluid supply line 128 via one or more connections 412 such that oxygen flowing in the fluid supply line 128 is distributed among the plurality of fluid transmission lines 404. The distal ends of the fluid transmission lines 404 are individually connected to a different oxygen output 132. Accordingly, the fluid transmission lines 404 act as a mechanism for distributing the oxygen received at the fluid supply line 128 among the plurality of oxygen outputs 132. The fluid supply line 128 may be used as a manifold or plenum from which the fluid transmission lines 404 and corresponding outputs 132 may run.

In one embodiment, the fluid transmission lines 404 and the fluid supply line 128 may be contained within the housing 104 of the device 400. Specifically, the housing 104 may comprise a first housing element 104a and a second housing element 104b. In some embodiments, the first housing element 104a may be manufactured separately from the second housing element 104b. The first and second housing elements 104a, 104b may comprise polymeric material, that is one or more of molded and machined. It is anticipated that the first and second housing elements 104a, 104b may be connected to one another by one or more of screws, fasteners, friction fittings, glue, welding, etc. In some embodiments, the first housing element 104a may be removably attached to the second housing element 104b to form the housing 104 of the device 400.

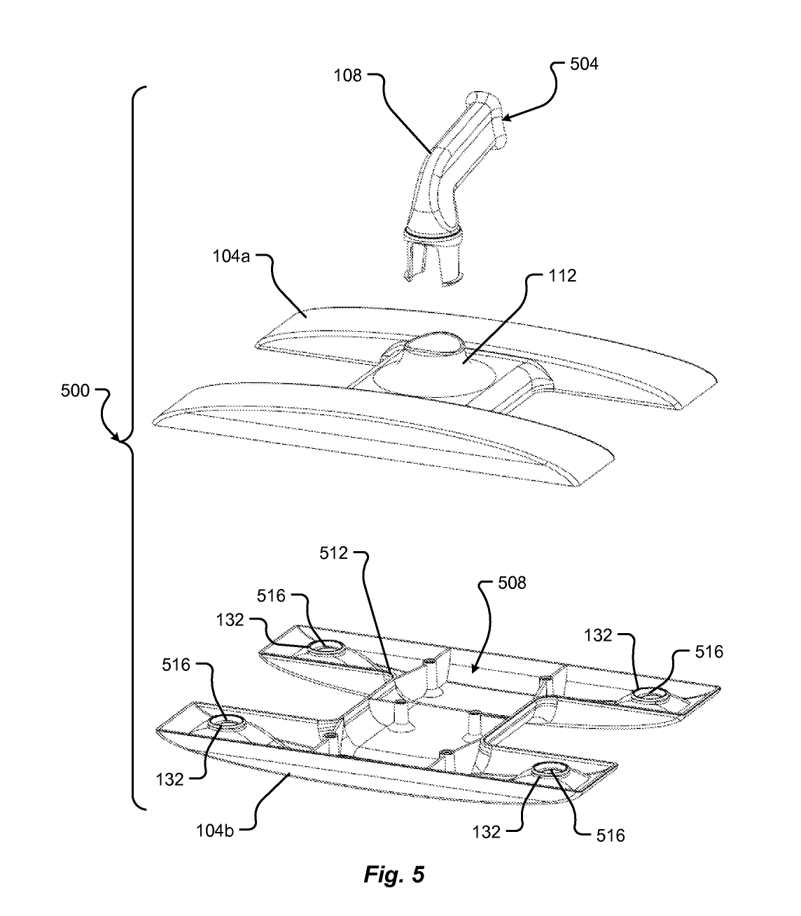

FIG. 5 is a second exploded perspective view of an oxygen delivery device 500 including a manifold or plenum area 508 in accordance with embodiments of the present disclosure. As illustrated, the oxygen from the fluid supply line 128 may be directed to a common manifold or plenum 508 where the oxygen outputs 132 are operatively connected to the manifold or plenum 508 area. This enables the fluid supply line 128 to directly distribute oxygen among the plurality of oxygen outputs 132 without requiring fluid transmission lines 404. Stated another way, the common manifold or plenum 508 may be used to contain the oxygen 136 in a central chamber and allow it to pass from this central chamber through the oxygen outputs 132. In some embodiments, a separate first housing element 104a and second housing element 104b may be removably attached to form the device 500 housing 104. Accordingly, the housing 104 may incorporate a gasket 512 disposed between at least one mating surface of the first housing element 104a and the second housing element 104b. The gasket 512 may be constructed from a compliant material that, when disposed between the first and second housing element 104a, 104b, is configured to compress and form an air tight area within the manifold or plenum 508 of the housing 104.

In some embodiments, the first and/or second housing element 104a, 104b may include features for receiving a gasket 512. The first and/or second housing elements 104a, 104b may also include one or more features for receiving and/or capturing fluid transmission lines 404 if such a mechanism is used. In yet another embodiment, the internal features of the housing elements 104a, 104b may be configured to accommodate the common manifold or plenum 508.

The second housing element 104b, or a plate connected to the housing 104, may comprise a number of openings 516 to accommodate the oxygen outputs 132. In some embodiments, the oxygen outputs 132 may comprise a plate fitting and a nozzle. The plate fitting may be used to connect the nozzle to the second housing element or plate. Additionally or alternatively, the oxygen outputs 132 may be contained at least partially within the housing 104.

In any embodiment described herein, the oxygen outputs 132 can be static, or adjustable for air flow and/or direction (similar to the nozzles described in issued U.S. Pat. No. 3,366,363; 5,127,876; 5,328,152; 5,399,119, each of which are hereby incorporated herein by reference in their entirety for all that they teach and for all purposes). In other words, the oxygen outputs 132 can remain static and uncontrollable for flow and/or direction, or be controlled with respect to direction and/or flow rate. It is anticipated that the best flow and direction for a particular patient could be achieved by employing a combination of both static and adjustable nozzles as oxygen outputs 132.

Additionally, the general oxygen flow rate of the device 100 could be controlled by a pressure or flow regulator operatively connected to the entrance or exit area of the manifold/plenum 508 or attached to the fluid supply line 128. This directed oxygen 136 could be controlled manually or automatically with an analog pressure regulator or via a digital controller and pressure regulator attached to or separate from the device 100. In some embodiments, the flow rate adjustment would be restricted to operate within a minimum and maximum range to ensure that a patient 124 is exposed to the ideal amount of oxygen required.

### EXAMPLE 1

The above-described system has been tested on at least one patient having cystic fibrosis. In a first experimental set, the patient was relatively healthy and the patient's pulse rate and oxygen saturation (SpO2) was measured for approximately 2 hours with only sleeping under environmental conditions (e.g., no oxygen delivery system employed). During the first experiment, the patient had a high SpO2 of 96%, a low SpO2 of 86%, and a mean SpO2 of 89.9%. Furthermore, it was determined that the patient's SpO2 was below 90% for 40.9% of the time.

During a second experiment one night following the first experiment, the same patient was placed within an oxygen-enriched environment (e.g., under an oxygen delivery system) for 1 hour and 15 minutes. During the second experiment, the patient had a high SpO of 98%, a low SpO2 of 88%, and a mean SpO2 of 93.7%. Moreover, it was determined that during the second experiment, the patient's SpO2 had dropped below 90% for only 1.3% of the time. As can be seen by comparing the first experiment with the second experiment, the patient's overall time with an SpO2 above 90% was dramatically increased.

### Example 2

In a third experimental set, the same patient of Example 1 was monitored similarly to the first experiment (e.g., in environmental air) except that the patient's health was compromised (e.g., the patient was feeling ill) and the experiment lasted three hours. In this experiment, the patient had a high SpO2 of 94%, a low SpO2 of 75%, and a mean SpO2 of 86.3%. It was calculated that the SpO2 of the patient was below 90% for 95.5% of the time.

In a fourth experimental set one night following the third experimental set, the same ill patient was placed within an oxygen-enriched environment for 1 hour and 21 minutes. During the fourth experiment, the patient had a high SpO2 of 98%, a low SpO2 of 86%, and a mean SpO2 of 92.3%. It was determined that during the fourth experiment the patient's SpO2 dropped below 90% for only 4.2% of the time. Thus, as can be seen from the above experimental results, the ill patient experienced significantly better oxygen saturation while using the oxygen delivery system described herein.

While various embodiments of the present disclosure have been described in detail, it is apparent that modifications and alterations of those embodiments will occur to those skilled in the art. However, it is to be expressly understood that such modifications and alterations are within the scope and spirit of the present disclosure, as set forth in the following claims. Further, the disclosure(s) described herein is capable of other embodiments and of being practiced or of being carried out in various ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

What is claimed is:

1. An oxygen delivery system comprising:
a housing having a top surface, a bottom surface, and at least two side surfaces, the bottom surface having a plurality of outputs distributed thereon and at least one input in fluid communication with the plurality of outputs via a fluid distribution mechanism contained within the housing;
an oxygen supply source;
a fluid supply line establishing a fluid connection between the oxygen supply source and the housing such that oxygen provided by the oxygen supply source is expelled away from the housing by the plurality of outputs thereby creating an oxygen-enriched environment outside of the housing.

2. The system of claim 1, wherein the fluid distribution mechanism comprises a common manifold positioned within the housing.

3. The system of claim 1, wherein the fluid distribution mechanism comprises a plurality of fluid transmission lines positioned within the housing, each of the fluid transmission lines connecting the fluid supply line with a respective output.

4. The system of claim 1, further comprising an arm and clamp system configured to connect the housing to at least one of the patient's bed, a wall, a ceiling, and an object near a patient.

5. The system of claim 1, wherein each output of the plurality of outputs is configured as a nozzle.

6. The system of claim 5, wherein the nozzle is configured for adjustment of at least one of position and fluid flow.

7. The system of claim 4, wherein the arm is configured to articulate about the connection to the housing.

8. The system of claim 7, further comprising:
a locking mechanism, wherein the locking mechanism is configured to lock at least one of the housing and the arm in a first position.

9. An oxygen delivery system, comprising:
an oxygen supply source;
a housing, comprising:
a volume with at least one outer surface, the volume including a cavity arranged within the volume, the cavity comprising at least one inner surface;
an input opening from the outer surface of the volume to the inner surface of the cavity, the input opening creating a first fluid path;
at least one output opening from the inner surface of the cavity to the outer surface of the volume, wherein the at least one output opening is different from the input opening, and wherein the at least one output opening creates a second fluid path;
a fluid supply line operatively connected to the input opening and establishing a fluid connection between the oxygen supply source and the housing such that oxygen provided by the oxygen supply source is directed away from the housing by the at least one output thereby creating an oxygen-enriched environment outside of the housing.

10. The system of claim 9, wherein the housing further comprises an arm connection arranged on the outer surface and configured to operatively connect to a support arm.

11. The system of claim 10, further comprising a support arm operatively connected to the arm connection.

12. The system of claim 11, wherein the support arm is configured to attach to a mechanical surface.

13. The system of claim 9, further comprising:
a fluid control valve operatively connected to the fluid supply line, wherein the fluid control valve is configured to adjust a flow of fluid presented to the input opening of the housing via the oxygen supply source.

14. The system of claim 13, further comprising:
a biometric sensor, the biometric sensor configured to monitor biological input provided by a patient.

15. The system of claim 14, wherein the biometric sensor is configured to provide a control signal based on the monitored biological input.

16. The system of claim 9, wherein the at least one output is configured as a nozzle.

17. The system of claim 16, wherein the nozzle is configured for adjustment of at least one of position and fluid flow.

18. A method of delivering supplemental oxygen to a patient in a contactless manner, comprising:
creating oxygen via a supply source;
directing the oxygen along a fluid supply line from the supply source to a fluid distribution system;
controlling the flow of oxygen from the distribution system through at least one nozzle, wherein the at least one nozzle is configured to direct the oxygen toward the patient located adjacent to the at least one nozzle;
causing the oxygen to mix with air at least partially surrounding the patient, thereby providing an oxygen-enriched environment in a volume at least partially surrounding the patient.

19. The method of claim 18, wherein a flow of oxygen directed along the fluid supply line is controlled via a fluid control valve.

20. The method of claim 18, wherein two or more nozzles are configured to be individually controlled for at least one of position and fluid flow.
